(19) United States
(12) Patent Application Publication
(10) Pub. No.: US 2024/0115417 A1
(43) Pub. Date: Apr. 11, 2024

(54) METHOD FOR PROVIDING THERMAL CONTRAST THERAPY

(71) Applicant: GENTHERM INCORPORATED, Northville, MI (US)

(72) Inventors: Nicola Gerrett, Royal Oak, MI (US); Emily Washington, Birmingham, MI (US); Nathaniel Gur-Arie, Ann Arbor, MI (US); Matthew Mallette, Paris (CA); Mark Powell, Northville, MI (US); Chad Pacilli, Windsor (CA)

(21) Appl. No.: 17/957,871

(22) Filed: Sep. 30, 2022

(51) Int. Cl. A61F 7/00; A61M 19/00; B60N 2/56

(52) U.S. Cl. CPC A61F 7/007; A61M 19/00; B60N 2/5678; A61F 2007/0071

(57) ABSTRACT

A method for providing thermal contrast therapy to a vehicle occupant. The method comprises first operating a heating device to apply a first temperature for a first period of time and second operating the heating device and a cooling device in an alternating manner for a plurality of time intervals. The thermal contrast therapy achieves about a 10° C. to 20° C. change in a temperature of a surface of a seat and/or about a 3° C. to 6° C. change in a skin temperature of the vehicle occupant.

### FIELD

The present disclosure relates to a method for providing thermal contrast therapy by a vehicle seat.

### BACKGROUND

Analgesia provided by vehicle seats has been proposed. Some have employed massaging devices in vehicle seats to address discomfort or even pain felt by occupants. Some vehicle seats include heating devices and/or cooling devices in addition to the massaging devices. However, massaging devices typically take up space within seats as well adding to the overall cost of the vehicle. Managing packaging space and costs are persistent concerns within the automotive industry.

Thermal therapy is a known procedure for providing analgesia. Generally, thermal therapy is administered by providing alternating heating and cooling to the body, often by way of immersion in a temperature-controlled water bath. Thermal therapy can be beneficial for pain reduction, injury treatment, and sport and exercise recovery. These effects can be attributed to increased blood flow within the targeted region by way of vasoconstriction and vasodilation influenced by cooling and heating periods, respectively. Moreover, acute application of heat can activate thermoreceptors and de-sensitize pain receptors. Other physiological outcomes may include decreased inflammation and/or increased range of motion.

Current clinical studies have investigated thermal therapy in general. However, operating parameters in vehicle environments, for heating and cooling devices in vehicle seats, have not been proposed. Moreover, time constraints in achieving peri-treatment and post-treatment effects commensurate with typical commute times (e.g., 15-25 minutes) have not been proposed. Current clinical studies have yet to investigate the role of skin temperature change magnitude and change rate in the therapy.

There is a need for a method for providing analgesia to vehicle occupants without the use of massaging devices.

There is a need for a method for providing analgesia to vehicle occupants using only existing heating and cooling devices in vehicle seats.

There is a need for a method for providing thermal therapy in vehicles.

There is a need for operating heating and cooling devices with specific time and temperature profiles to provide occupants peri-treatment and/or post-treatment effects.

There is a need for operating heating and cooling devices to target specific regions of occupants' bodies.

There is a need for providing thermal therapy that accounts for clothing typically worn by occupants in warm and cold seasons.

### SUMMARY

The present disclosure describes a method for providing thermal contrast therapy to a vehicle occupant, which may address at least some of the needs identified above. The method may comprise first operating a heating device to apply a first temperature for a first period of time and second operating the heating device and a cooling device in an alternating manner for a plurality of time intervals.

Operating the heating device to the first temperature for the first period of time may bring a temperature of at least one section of a seat surface to about 43° C. to 48° C. and/or a skin temperature of the vehicle occupant in a region of the at least one section to about 36° C. or more.

During the plurality of time intervals, the heating device may be operative for a second period of time. The second period of time may be about 3 to 5 minutes. During the plurality of time intervals, the cooling device may be operative for a third period of time. The third period of time may be about 3 to 6 minutes.

The thermal contrast therapy may achieve about a 10° C. to 20° C. change in the temperature of the at least one section of the seat surface and/or about a 3° C. to 6° C. change in the skin temperature of the vehicle occupant in the region of the at least one section of the seat surface.

During the plurality of time intervals, the heating device and the cooling device may effectuate temperature changes in the at least one section of the seat surface. The at least one section may include a lower section, a middle section, an upper section, or any combination thereof.

The heating device may be located in the lower and middle sections and during the plurality of time intervals the heating device in the lower section may be brought to a target temperature of about 63° C. to 67° C. (e.g., 65° C.) and the heating device in the middle section may be brought to a target temperature of about 77° C. to 83° C. (e.g., 80° C.).

The heating device may be located in the upper section and during the plurality of time intervals the heating device in the upper section may be brought to a target temperature of about 67° C. to 73° C. (e.g., 70° C.).

During the plurality of time intervals, the cooling device may be brought to a target temperature of about 18° C. to 22° C. (e.g., 20° C.).

During the plurality of time intervals, the heating device may be operated by pulse width modulation with a duty cycle of about 55% to 65% (e.g., 60%). During the plurality of time intervals, the cooling device may be operated by pulse width modulation with a duty cycle of about 85% to 95% (e.g., 90%).

The operating time of the heating device and the cooling device, during the plurality of time intervals, may be equal.

The rate of temperature change during the plurality of time intervals may be ±0.4° C./minute to ±0.9° C./minute for the skin temperature and/or ±1° C./minute to ±2.5° C./minute for the seat temperature.

The plurality of time intervals may persist for a duration of about 20 to 35 minutes.

The alternating heating and cooling may be respectively characterized by a ramp-and-step profile. The ramp-and-step profile of heating may be inverse to the ramp-and-step profile of cooling.

There may be no idle period between the alternating operation of the heating device and the cooling device. The idle period may be characterized by a period of time in which both the heating device and the cooling device are not operational.

The heating device may be operated by pulse width modulation with a duty cycle of about 55% to 65% (e.g., 60%) to realize the first temperature.

The first temperature may be about 55° C. to 90° C.

The first period of time may be about 5 to 10 minutes.

The time intervals may include 6 to 16 time intervals.

The thermal contrast therapy may be adapted to relieve pain of the vehicle occupant.

The at least one section of the seat surface may include a lower section and/or a middle section.

The present disclosure describes a device which may address at least some of the needs identified above. The device may perform the method described above. The device may comprise the heating device located in an upper section of the seat, a middle section of the seat, and a lower section of the seat; and a cooling device acting upon the middle section of the seat.

The heating device may comprise a resistance element. The cooling device may comprise a blower, a thermoelectric device, a fluid distribution device, or any combination thereof.

The heating device in the lower section may have a surface power density of about 2,100 to 2,500 W/m². The heating device in the middle section may have a surface power density of about 1,900 to 2,300 W/m². The heating device in the upper section may have a surface power density of about 900 to 1,500 W/m².

The present disclosure describes a vehicle seat which may address at least some of the needs identified above. The vehicle seat may comprise the device described above.

### BRIEF DESCRIPTION OF THE DRAWINGS

FIG. 1 is a schematic of a vehicle seat.

FIG. 2A is a graph of skin temperature over time during the control trial according to the present teachings.

FIG. 2B is a graph of seat surface temperature over time during the control trial according to the present teachings.

FIG. 3A is a graph of heater and cooler target temperatures over time during the experimental trial according to the present teachings.

FIG. 3B is a graph of heater and cooler duty cycles over time during the experimental trial according to the present teachings.

FIG. 3C is a graph of skin temperature over time during the experimental trial according to the present teachings.

FIG. 3D is a graph of seat surface temperature over time during the experimental trial according to the present teachings.

FIG. 3E is a graph of changes in skin temperature over different time intervals during the experimental trial according to the present teachings.

FIG. 3F is a graph of changes in seat surface temperature over different time intervals during the experimental trial according to the present teachings.

FIG. 4A is a graph of subjective pain for the control trial.

FIG. 4B is a graph of subjective pain for the experimental trial.

### DESCRIPTION

### Introduction

The present teachings relate to a method for providing thermal contrast therapy, a device for providing the thermal contrast therapy, and a vehicle seat comprising the device. The thermal contrast therapy may alternate heating and cooling of a human subject (e.g., a vehicle occupant). The thermal contrast therapy may be applied to the backs of human subjects.

The thermal contrast therapy may be applied by one or more heating devices and cooling devices. The target temperatures and the operational durations of the heating devices and the cooling devices may be selected to modulate the temperature of a seat surface, modulate the skin temperature of a human subject, realize a clinically significant effect (reduction) in the pain levels of human subjects, ensure a temperature difference is felt by the human subjects, or any combination thereof.

The target temperatures and the operational durations of the heating devices and the cooling devices may be selected to realize ratios of heating periods to cooling periods, temperature gradients, seat surface temperature change rates, human subject skin temperature change rates, or any combination thereof.

The target temperatures and operational durations of the heating devices and cooling devices may be modulated commensurate with seasonal and/or regional climates, which may vary in ambient temperature, humidity, and the like. That is, the thermal insulation of clothing worn during different seasons and/or in different regions may be accounted for. By way of example, the target temperature and the operational duration during fall and winter months, where thicker clothing layers are typically worn, may be different relative to spring and summer months, where thinner clothing layers are typically worn. Moreover, the present method may account for regions in which vehicles are located. By way of example, a seasonal difference in the thermal insulation of clothing layers may not be accounted for in regions with tropical climates due to relatively constant weather conditions throughout the year, but the same may be accounted for in regions with continental climates, where weather fluctuates throughout the year.

The thermal contrast therapy may provide pain relief (“analgesia”) to the human subjects (e.g., vehicle occupants). Analgesia may be realized during thermal contrast therapy and/or after thermal contrast therapy. Analgesia may be realized 0.5 hours or less, 1 hour or less, 2 hours or less, 3 hours or less, or even 6 hours or less after thermal contrast therapy.

The thermal contrast therapy may be directed to the backs, buttocks, or thighs of the human subjects, or any combination thereof. The thermal contrast therapy may be directed to the upper back, middle back, lower, back, or any combination thereof. It may be advantageous to apply thermal contrast therapy to at least the lower back, which is most often afflicted by pain, whether acute or chronic. Moreover, the thermal contrast therapy of the present teachings may be applied by a seat (e.g., a vehicle seat) on which pressure applied by occupants may be concentrated in the lower back and/or middle back.

Any part of the body and/or portions thereof may be selected by human subjects to be targeted by the thermal contrast therapy. One or more parts of the body and/or portions thereof may be targeted. Multiple parts of the body and/or portions thereof may be brought to the same or different skin temperatures.

Habitual application of thermal contrast therapy (e.g., during morning and/or evening daily work commutes) may provide compounded benefits to the human subjects. That is, the magnitude of the change in pain level (relative to pain levels realized without the thermal contrast therapy) during and/or after thermal contrast therapy may increase in proportion to the frequency of use. Moreover, the duration over which reduced pain (relative to pain levels realized without the thermal contrast therapy) is realized after the cessation of thermal contrast therapy may increase in proportion to the frequency of use.

While the present disclosure discusses therapy durations in the context of typical vehicle commute times, the present disclosure contemplates that the thermal contrast therapy may persist for lesser or greater periods of time. By way of example, the thermal contrast therapy may be applied for 0.5 hours or more, 1 hour or more, 2 hours or more, or even 3 hours or more during long trips. The present disclosure that the thermal contrast therapy may be applied multiple times during a single trip. Multiple therapy iterations may or may not be separated by a period of time in which therapy is not applied.

Device.

The thermal contrast therapy may be administered in vehicles. Vehicles may include one or more seats comprising heating devices and/or cooling devices. The heating devices and/or cooling devices may be operated to pre-determined target temperatures and operational time periods to provide thermal contrast therapy according to the present teachings. One or more heating and/or cooling devices may be located in different sections of a vehicle seat and thus apply the thermal contrast therapy to different regions of an occupant's body.

The vehicle seat may comprise a back portion and a seat portion. The back portion and/or seat portion may comprise one or more heating devices and/or one or more cooling devices. One or more heating and/or cooling devices may be located in one or more sections of the vehicle seat. Each seat section may be contacted by a different body region of an occupant. A single heating and/or cooling device may comprise one or more zones. The zones may be capable of operating independently of each other. The one or more zones may be located in one or more sections of the vehicle seat.

The back portion and/or seat portion may comprise one or more bolsters. The bolsters may extend at least partially beyond the body profile of an occupant. The bolsters may extend at an angle relative to a seating surface and/or back rest surface. The bolsters may function to at least partially mitigate lateral movement of occupants while driving (e.g., while driving around corners). One or more regions of an occupant's body may contact one or more bolsters. One or more regions of an occupant's body may exchange heat with one or more bolsters.

The lower, middle, and upper sections referred to herein may be of the back portion of the vehicle seat. Generally, the lower section extends across the region of the back having the lumbar vertebrae segment, the middle section extends across the region of the back having the T7-T12 vertebrae segment, and the upper section extends across the region of the back having the T1-T6 vertebrae segment. The present disclosure contemplates that occupants may have different body dimensions and thus, skilled artisans can adapt the present teachings such that the divisions between the lower, middle, and upper sections described above may vary accordingly.

The heating devices and/or cooling devices may be located under a surface of the vehicle seat. One or more layers of material (e.g., fabric, foam, film, and the like) may be disposed between the heating and/or cooling devices and the surface of the seat. The heating devices and/or cooling devices may provide thermal sensation to occupants via conduction and/or convection. With conduction, heat may be conducted through one or more layers of the vehicle seat and ultimately to an occupant contacting the surface of the vehicle seat. With convection, heated and/or cooled air may travel through one or more layers of the vehicle seat and the air may contact the occupant.

The heating devices may comprise one or more resistance elements. The resistance elements may have a negative temperature coefficient, a positive temperature coefficient, a constant temperature coefficient, or any combination thereof. The resistance elements may heat when a current is applied thereto. The resistance elements may be carried on a medium. The medium may include a film, a nonwoven mat, a woven mat, or any combination thereof.

An exemplary heating device is described in U.S. Pat. No. 9,315,133 B2, incorporated herein by reference in its entirety for all purposes.

The heating devices may be adapted to the section of the vehicle seat within which they reside. The heating devices in different sections of the vehicle seat may have different surface power densities. Higher surface power densities may be provided to a lower and/or middle section of a seat back relative to an upper section of a seat back. In this regard, thermal contrast therapy may be concentrated in the lower and middle sections, understanding that these regions of the back are commonly afflicted with acute and/or chronic pain. The present teachings contemplate that all sections of the seat have equal surface power density or that the upper section has a greater surface power density relative to the lower and/or middle sections.

A heating device located in a lower section of a seat back may have a surface power density of about 1,100 W/m² or more, 1,300 W/m² or more, 1,500 W/m² or more, or even 1,700 W/m² or more. A heating device located in a lower section of a seat back may have a surface power density of about 2,500 W/m² or less, 2,300 W/m² or less, 2,100 W/m² or less, or even 1,900 W/m² or less.

A heating device located in a middle section of a vehicle seat back may have a surface power density of about 1,100 W/m² or more, 1,300 W/m² or more, 1,500 W/m² or more, or even 1,700 W/m² or more. A heating device located in a middle section of a seat back may have a surface power density of about 2,300 W/m² or less, 2,100 W/m² or less, or even 1,900 W/m² or less.

A heating device located in an upper section of a vehicle seat back may have a surface power density of about 900 W/m² or more, 1,000 W/m² or more, or even 1,100 W/m² or more. A heating device located in an upper section of a vehicle seat back may have a surface power density of about 1,500 W/m² or less, 1,400 W/m² or less, or even 1,300 W/m² or less.

A heating device located in bolsters of a vehicle seat may have a surface power density of about 800 W/m² or more, 900 W/m² or more, or even 1,000 W/m² or more. A heating device located in bolsters of a vehicle seat may have a surface power density of about 1,400 W/m² or less, 1,300 W/m² or less, or even 1,200 W/m² or less.

The power densities, discussed above, may be the maximum power densities of the heating devices. As discussed herein, the duty cycle may be less than 100%. Thus, the effective power densities of the heating devices, during operation, may be less than the maximum power densities, in accordance with the duty cycle realized during operation. By way of example, a heating device with a 2,400 W/m² maximum power density may have an effective power density of 1,200 W/m² when operating at a 50% duty cycle.

The surface power density of the heating device in the lower section may be greater than the surface power density of the heating device in the middle section by about 5% or more, 7% or more, or even 9% or more. The surface power density of the heating device in the lower section may be greater than the surface power density of the heating device in the middle section by about 15% or less, 13% or less, or even 11% or less. The surface power density of the heating device in the lower section may be generally equal to the surface power density of the heating device in the middle section.

The surface power density of the heating device in the middle section and/or lower section may be greater than the surface power density of the heating device in the upper section by about 40% or more, 45% or more, or even 50% or more. The surface power density of the heating device in the middle section and/or lower section may be greater than the surface power density of the heating device in the upper section by about 65% or less, 60% or less, or even 55% or less.

The cooling devices may comprise one or more blowers, conduits, fluid distribution devices, thermoelectric devices, or any combination thereof.

The blower may intake air (e.g., from underneath and/or behind a vehicle seat) and deliver air to one or more fluid distribution devices. The blower may be an axial fan or a radial fan. Non-limiting examples of blowers are described in International Publication No. WO 2008/115831 A1 and U.S. Pat. No. 9,121,414 B2, incorporated herein by reference for all purposes.

One or more conduits may extend between the blower and one or more fluid distribution devices. The one or more conduits may carry air between the blower and the one or more fluid distribution devices.

The fluid distribution devices may be located underneath a surface of a vehicle seat. One or more layers of material (e.g., fabric, foam, film, and the like) may be disposed between the fluid distribution devices and the surface of the vehicle seat.

The fluid distribution devices may comprise an enclosure (e.g., a bag). The enclosure may be defined by one or more segments of material. By way of example, two segments of material may be fastened around a perimeter of the enclosure by a seam. By way of another example, the enclosure may be defined by a single segment of material.

Air delivered from one or more blowers may enter the enclosure. The enclosure may be at least partially hermetically sealed. The enclosure may comprise one or more openings. Air may exit the enclosure via the one or more openings. The one or more openings may deliver air to a surface of a vehicle seat.

The fluid distribution devices may conductively and/or convectively thermally communicate with an occupant. Heated or cooled air residing within the enclosure may conductively thermally communicate with the walls of the enclosure, the walls may conductively thermally communicate with one or more layers of material in a seat disposed between the enclosure and the seat surface, the seat surface may conductively thermally communicate with an occupant, or any combination thereof. Heated or cooled air may pass through the enclosure via one or more openings, pass through one or more openings extending through one or more layers of the seat and/or seat surface, convectively thermally communicate with an occupant, or any combination thereof. One or more openings may expel air to a location other than the surface of the seat. One or more openings may be part of a fluid circuit, delivering air back to the inlet of the blower.

The fluid distribution devices may be located in a lower, upper, or middle section of a vehicle seat back, or any combination thereof. The fluid distribution devices may be located in seat sections where pressure is applied by occupants. The fluid distribution devices may expel air from a lower, upper, or middle section of a vehicle seat back, or any combination thereof.

Air delivered to the enclosure may be heated and/or cooled. The air may be heated and/or cooled by one or more thermoelectric devices. The one or more thermoelectric devices may be located in one or more blowers, conduits, enclosures, or any combination thereof.

The thermoelectric devices may be Peltier devices. The thermoelectric devices may comprise two surfaces, one of which being the “hot” side and the other being the “cold” side, relative to each other. By reversing the polarity of power provided to the thermoelectric device, the relative temperatures of each side may flip. A non-limiting example of a thermoelectric device is described in U.S. Pat. No. 9,857,107 B2, incorporated herein by reference for all purposes.

The cooling device may adjust operation to provide heat that is supplemental to the heating device. In this regard, the polarity of one or more thermoelectric devices may be switched to change the cold side thereof to the hot side.

Method.

The present disclosure describes a method for providing thermal contrast therapy to a vehicle occupant. The thermal contrast therapy may be adapted to relieve pain of the vehicle occupant. Pain may be relieved peri-treatment and/or post-treatment.

The method of the present disclosure may comprise operating a heating device to apply a first temperature for a first period of time. The first temperature may be about 55° C. to 90° C. The first period of time may be about 5 to 10 minutes. Operating the heating device to the first temperature during the first time may overcome a thermal inertia of a vehicle seat and/or raise the skin temperature of an occupant to therapeutic levels. The temperature during the first time may be greater than the temperature during the plurality of time intervals, discussed below.

Temperatures of the heating device, as referred to herein, may mean the temperature of the resistance element (e.g., negative temperature coefficient resistance element).

The heating device may be operated by regulating power provided thereto. Power may be regulated by pulse width modulation (PWM), constant current control, or the like. The heating device may be provided a duty cycle of at least about 60%, at least about 70%, at least about 80%, at least about 90% power, or even 100% to realize the first temperature.

The heating device may be operated during the first time to bring a temperature of at least one section of a seat surface to about 43° C. to 48° C. and/or a skin temperature of a vehicle occupant to about 34° C. to 40° C. Preferably, skin temperature is brought to about 36° C. or more, which is considered therapeutic, but less than the pain/burn threshold. The at least one section may include a lower, middle, and/or upper section. Preferably the at least one section may include a lower and/or a middle section. The skin temperature of the vehicle occupant may be the skin temperature of the portion of the skin contacting (directly, or indirectly with clothes disposed between the seat surface and skin) the lower, middle, and/or upper section.

The therapy may be applied to an occupant's spine and/or an area proximate to the spine. The therapy may be applied across the back, at least partially from one side of the back to an opposing side of the back, and in sections delineated at different heights along the spine.

A temperature of 43° C. is conventionally considered the threshold at which occupants may experience pain and a temperature of 44° C. is conventionally considered the threshold at which occupants may experience a burn. However, while the seat surface temperature exceeds these pain/burn thresholds, the time during which the pain/burn thresholds are exceeded (e.g., 2 minutes or less), the rate of temperature change (e.g., no more than 0.5° C./minute, according to Carter et al., Sensory and sympathetic nerve contributions to the cutaneous vasodilator response from a noxious heat stimulus, Exp. Physiol. 96.11:1208-1217), the thermal insulation of the participants' clothing (e.g., at least about 0.05 Clo), or any combination thereof may preclude a pain/burn response in the participants.

The method may comprise operating the heating device and a cooling device in an alternating manner for a plurality of time intervals. The quantity of time intervals may include 6 or more, 8 or more, or even 10 or more time intervals. The quantity of time intervals may include 16 or less, 14 or less, or even 12 or less time intervals.

During each of the time intervals the heating device may be operative for about 3 to 5 minutes. During each of the time intervals the cooling device may be operative for about 3 to 6 minutes. The operating time of the heating device and the cooling device, during the plurality of time intervals, may be equal or unequal. The operating time of the heating device and the cooling device may or may not overlap. There may be a duration of time in each time interval in which either the heating or cooling device is operational, and the other device is not operational.

The plurality of time intervals may persist for a duration of about 20 to 35 minutes, or until the operation is terminated. The vehicle occupant may manually terminate the operation. A fault condition may automatically terminate the operation (e.g., overheating beyond a threshold temperature and/or a threshold temperature being surpassed for a pre-determined period of time).

There may or may not be an idle period between the alternating operation of the heating device and the cooling device. An idle period may be characterized by a period of time in which both the heating device and the cooling device are not operational.

The alternating heating and cooling may be respectively characterized by a ramp-and-step profile. The ramp-and-step profile may present as a steep increase in temperature, holding temperature for a period of time, and a steep decrease in temperature. The ramp-and-step profile of heating may be inverse to the ramp-and-step profile of cooling.

The thermal contrast therapy may achieve about a ±10° C. to ±20° C. change in a temperature of a surface of a seat and/or about a ±3° C. to ±6° C. change in the skin temperature of the vehicle occupant. The magnitude of the change may promote vasodilation and vasoconstriction in occupants.

The rate of temperature change during the plurality of time intervals may be about (±0.2) ±0.4° C./minute to ±0.9° C./minute for the skin temperature and/or about (±0.2) ±1° C./minute to ±2.5° C./minute for the seat temperature. These temperature change rates may apply in environments, outside and/or within the vehicle, with an air temperature of about 20° C. to 25° C. In environments with an air temperature of below 20° C., the temperature change rate for the skin temperature may be up to about ±1.8° C./minute. In environments with an air temperature of below 20° C., the temperature change rate for the seat temperature may be up to about ±5° C./minute.

The present disclosure contemplates that a specific change magnitude and/or change rate in skin temperature may be sought to provide the intended thermal sensation to the vehicle occupant. That is, the present method seeks to provide thermal contrast therapy that is perceivable by vehicle occupants while also keeping vehicle occupants comfortable, understanding that while uncomfortable thermal contrast therapy may be effective in relieving pain, occupants may be dissuaded from using a vehicle's thermal therapy feature that causes discomfort. By way of example, a relatively fast change rate may cause pain to an occupant while a relatively slow change rate may not be perceived by an occupant.

Thermal sensations (i.e., comfort levels) may not solely depend on the temperature of a surface that is contacted by skin, but more notably may depend on the temperature change rate of the skin.

The present method may be controlled and/or benchmarked based on the temperature of a heating device, a cooling device, and/or a seat surface, but ultimately these temperatures may be related to how they influence temperature change magnitudes and/or change rates of occupants' skin.

The heating device and the cooling device may effectuate temperature changes in at least one section of the surface of the seat. The at least one section may include a lower section, a middle section, an upper section, or any combination thereof. Preferably the at least one section includes the lower section and optionally the middle section, where back pain is commonly located and where pressure against vehicle seats is typically concentrated.

During the plurality of time intervals, a heating device located in a lower section of a vehicle seat may be brought to a target temperature of about 60° C. or more, 63° C. or more, or even 65° C. or more. A heating device located in a lower section of a vehicle seat may be brought to a target temperature of about 71° C. or less, 69° C. or less, or even 67° C. or less.

During the plurality of time intervals, a heating device located in a middle section of a vehicle seat may be brought to a target temperature of about 74° C. or more, 77° C. or more, or even 80° C. or more. A heating device located in a middle section of a vehicle seat may be brought to a target temperature of about 89° C. or less, 86° C. or less, or even 83° C. or less.

During the plurality of time intervals, a heating device located in an upper section of a vehicle seat may be brought to a target temperature of about 64° C. or more, 67° C. or more, or even 70° C. or more. A heating device located in an upper section of a vehicle seat may be brought to a target temperature of about 79° C. or less, 76° C. or less, or even 73° C. or less.

The target temperature of the lower and middle sections relative to the upper section may be based on typical pressures applied to those sections by occupants and/or where back pain is commonly located. Typically, greater pressures are realized in the lower and middle sections relative to the upper section.

During the plurality of time intervals, the cooling device may be brought to a target temperature of about 16° C. or more, 18° C. or more, or even 20° C. or more. During the plurality of time intervals, the cooling device may be brought to a target temperature of about 26° C. or less, 24° C. or less, or even 22° C. or less.

During the plurality of time intervals, the heating device may be operated by regulating power provided thereto. Power may be regulated by pulse width modulation (PWM), constant current control, or the like. The heating device may be provided a duty cycle of about 50% or more, 55% or more, or even 60% or more. The heating device may be provided a duty cycle of about 75% or less, 70% or less, or even 65% or less. The cooling device may be provided a duty cycle of about 75% or more, 80% or more, or even 85% or more. The cooling device may be provided a duty cycle of about 100% or less, 95% or less, or even 90% or less.

The method may be controlled based on time, temperature, or both. Operation of one or more heating devices and/or cooling devices may begin and terminate when a pre-determined time is met and/or when a pre-determined temperature is met.

The method may be controlled based on sensor feedback. The sensor feedback may provide the temperature of one or more heating devices and/or cooling devices. Each section of a heating device may comprise a temperature sensor. That is, different resistance element circuits that are independently controllable may each comprise a sensor. The sensor feedback may provide the temperature of a seat surface and/or an occupant's skin.

The method may be controlled based upon a dynamically estimated seat surface temperature and/or skin temperature.

### Example

Summary.

A human trial was performed using the thermal contrast therapy according to the present teachings. During the trial seat surface and participant skin temperature were monitored and participants were surveyed regarding their subjective local thermal temperature sensation levels, subjective thermal comfort level, and subjective pain level. The human trial showed that the thermal contrast therapy provided clinically significant benefit to participants.

Apparatus.

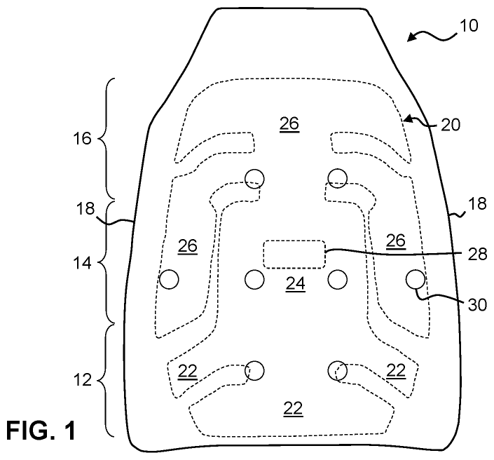

A vehicle seat 10 was constructed according to the schematic in FIG. 1. The vehicle seat 10 comprises a lower section 12, a middle section 14, an upper section 16, and two side bolsters 18. A heating device 20 is disposed under the surface of the vehicle seat 10 and comprises a first zone 22, a second zone 24, and a third zone 26. The first zone 22 has a surface power density of 2,407 W/m², the second zone 24 has a surface power density of about 2,142 W/m², and the third zone 26 has a surface power density of about 1,101 W/m². The vehicle seat 10 comprises a cooling device 28 located in the middle section 15 of the vehicle seat 10 and within the boundaries of the second zone 24 of heating device 20. Eight sensors 30 for measuring temperature were placed at different locations on the surface of the vehicle seat 10.

Participant skin temperature was monitored at 9 sites across the back in approximately a 3×3 grid arrangement. A sensor was located over the spine at the lumbar vertebrae segment, the lower thoracic vertebrae segment (within the T7-T12 vertebrae region), and the upper thoracic vertebrae segment (within the T1-T6 vertebrae region). Moreover, proximate to each vertebrae segment, two additional sensors were located on either side of the spinal sensors.

During the trial, participants reported their subjective local thermal sensation levels at each of the 9 sites across their backs (ranked for each of the 9 sites by 9 sensation levels from very hot (level 4) to very cold (level −4)), subjective local thermal comfort levels (ranked by 8 comfort levels from extremely comfortable (level 4) to extremely uncomfortable (level −4)), and subjective pain level (ranked by 11 pain levels from no pain (rank 0) to worst pain imaginable (rank 10)). After the trial, participants reported their subjective pain over a 2-hour period to determine to what extent, if any, the thermal therapy provided prolonged effects after cessation thereof.

Regarding the subjective pain level, Chou et al., Nonpharmacologic Therapies for Low Back Pain: A Systematic Review for an American College of Physicians Clinical Practice Guideline, Ann. Int. Med. (2017) provides guidelines for classifying changes in pain levels. Any change in pain level less than 0.5 is associated with no effect, any change in pain level of 0.5 to 0.9 is associated with a small effect, any change in pain level of 1 to 2 is associated with a moderate effect (classified as clinically significant), and any change in pain level greater than 2 is associated with a large effect (also classified as clinically significant). Chou states that most medications (e.g., anti-inflammatory, relaxants, opioids, etc.) typically prescribed for low back pain are associated with small to moderate and short-term effects.

Participants.

The trial was conducted on 15 participants ranging from 20 to 70 years in age (average age of 45), 9 of whom were female and 6 of whom were male. The participants wore clothes having a thermal insulation ranging from 0.29 to 0.45 Clo (1 Clo=0.155 m² K/W) (average of 0.38 Clo). Prior to the trial, 5 participants reported chronic back pain (i.e., persisting greater than 12 weeks), 5 participants reported sub-acute back pain (i.e., persisting for 4 to 12 weeks), and 5 participants reported acute back pain (i.e., persisting for less than 4 weeks).

Methodology.

In a control trial, participants were seated for 33 minutes during which time no operation of the heating device or the cooling device occurred.

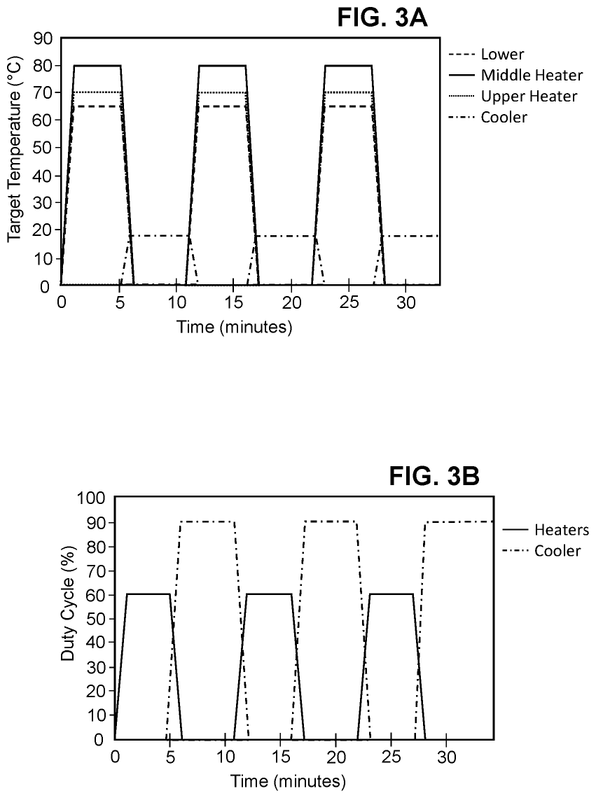

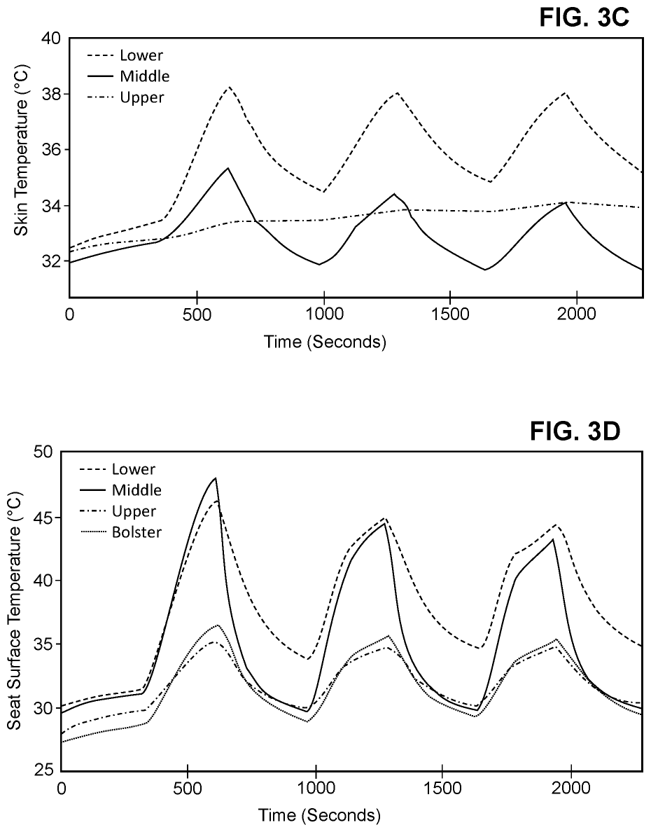

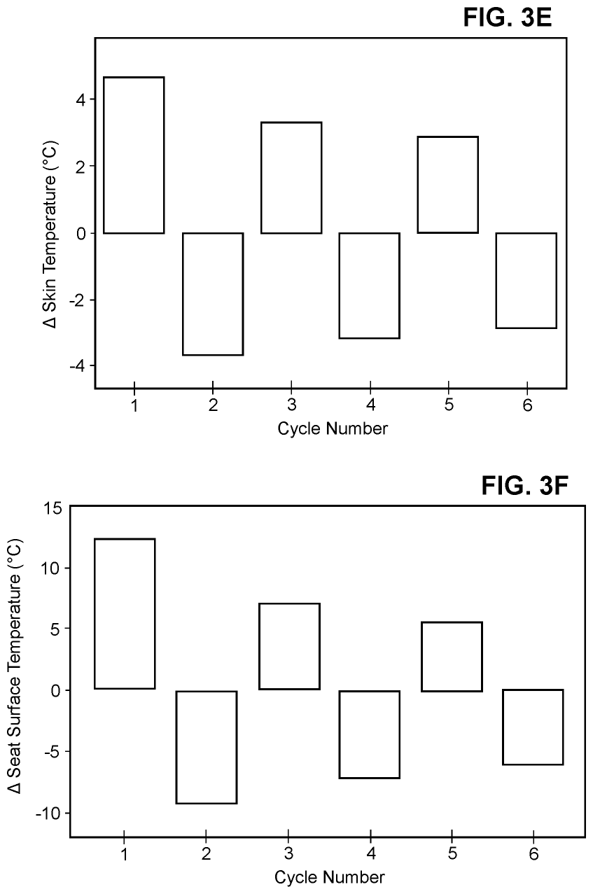

In the experimental trial, participants were seated and preconditioned for 5 minutes during which time no operation of heating device or the cooling device occurred. Then, thermal contrast therapy was applied to the seated participants for 33 minutes. During the thermal contrast therapy, the first and second zones of the heating device in the lower and middle sections of the seat were operated to a target temperature of 65° C., the third zone of the heating device in the upper section of the seat was operated to a target temperature of 80° C., the time to target temperature was 1 minute, and the target temperature was held for 4 minutes. Then operation of the heating device ceased. At the time the heating device ceased operation, the cooling device began operation. The cooling device was operated to a target temperature of 20° C., the time to target temperature was 1 minute, and the target temperature was held for 5 minutes. Then the operation of the cooling device ceased, and the time interval repeated for a total of 3 heating and cooling time intervals. The profile of this heating and cooling is referred to herein as a ramp-and-step profile. The target temperature and the duty cycle of the heating device and the cooling device to achieve the target temperatures are illustrated in FIGS. 3A-3B.

Results.

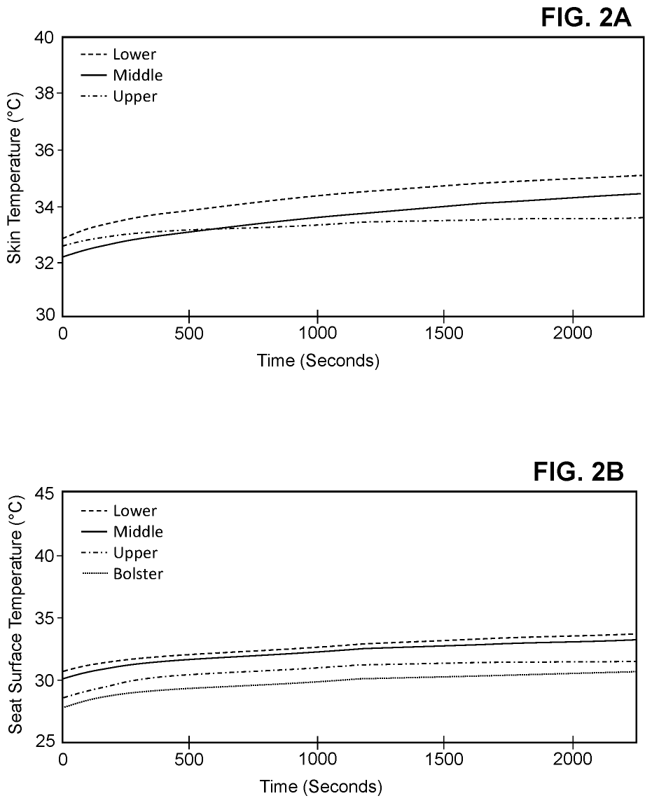

In the control trial, the participants' skin temperature and the seat surface temperature were monitored and are illustrated in FIGS. 2A-2B (expressed as an average of all participants). The participants' skin temperature increased on average by about 1.5° C., due to heat transfer between participant and seat via conduction, and the seat surface temperature remained on average about 2° C. below skin temperature. Thermal sensation level remained on average neutral (level 0) over the duration of the trial; thermal comfort remained on average as slightly comfortable (level 1) over the duration of the trial; and mean subjective pain on average did not change as measured from immediately prior to the trial to the end of the 2-hour period starting after the trial.

In the experimental trial, the participants' skin temperature and seat surface temperature were monitored and are illustrated in FIGS. 3C-3D (expressed as an average of all participants). In accordance with the target temperatures of the heating device and the cooling device illustrated in FIG. 3A, the participant skin temperature and the seat surface temperature increase to a sharp peak during heating and decrease to a sharp valley during cooling. Skin temperature fluctuated on average by about ±3.5° C. in the lower and middle back region. Lack of fluctuation in the upper back may be attributed to lack of contact and/or pressure of the participant against the seat surface. The seat surface temperature fluctuated on average by about ±10-20° C.

The rates of change in temperature of the participants' skin and the seat surface in each of the time intervals (expressed as an average of all participants) are provided in Table 1, below.

TABLE 1

| Rate of Change | Heating | Cooling | Heating | Cooling | Heating | Cooling |
|---|---|---|---|---|---|---|
| (° C./min) | Event 1 | Event 1 | Event 2 | Event 2 | Event 3 | Event 3 |
| Skin | 0.93 | −0.62 | 0.66 | −0.52 | 0.57 | −0.49 |
| Seat | 2.4 | −1.5 | 1.4 | −1.2 | 1.2 | −1.0 |

In the experimental trial, the seat surface temperature exceeded 43° C. for an average of 160 seconds in event 1, 180 seconds in event 2 and 120 seconds in event 3. A temperature of 43° C. is conventionally considered the threshold at which occupants experience pain. However, while the seat surface temperature exceeds this pain threshold, the time during which the pain threshold is exceeded, the rate of temperature change, and the thermal insulation of the participants' clothing preclude pain response in the participants.

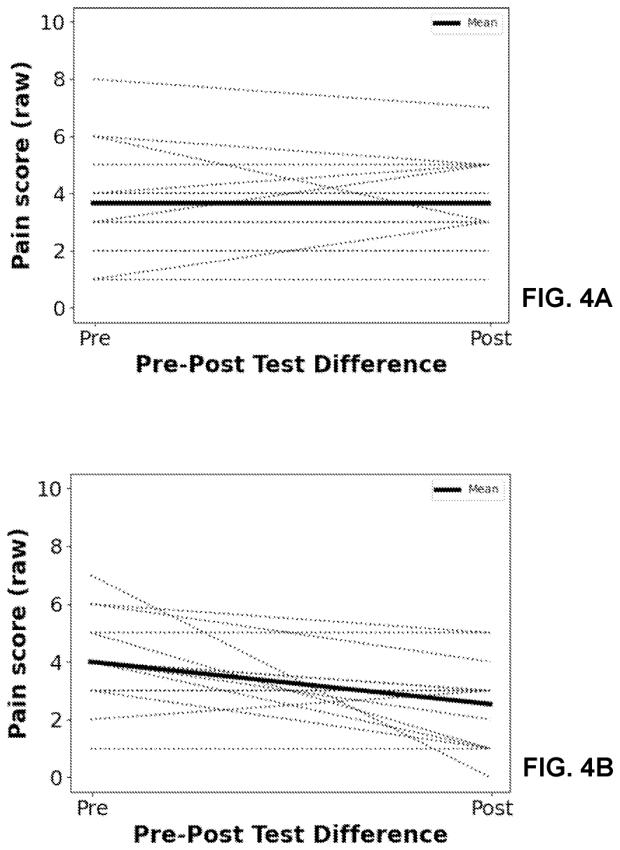

In the experimental trial, average thermal sensation levels fluctuated between hot (level 3) and cold (level −3) over the duration of the trial; average thermal comfort fluctuated between comfortable (level 2) during hot events and slightly comfortable (level 1) during cold events over the duration of the trial; and average subjective pain decreased by 1.5 (i.e., moderate effect) as measured from immediately prior to the trial to the end of the 2-hour period starting after the trial, with the decline in subjective pain being most prevalent during the first 25 minutes of the thermal contrast therapy. 60% of participants reported a reduction in pain. Subjective pain as measured pre-trial and post-trial for the control trial is illustrated in FIG. 4A. Subjective pain as measured pre-trial and post-trial for the experimental trial is illustrated in FIG. 4B. The reporting from individual subjects is represented by the dashed lines and the average over all reporting participants is represented by the solid line.

The explanations and illustrations presented herein are intended to acquaint others skilled in the art with the invention, its principles, and its practical application. The above description is intended to be illustrative and not restrictive. Those skilled in the art may adapt and apply the invention in its numerous forms, as may be best suited to the requirements of a particular use. Other combinations are also possible as will be gleaned from the following claims, which are also hereby incorporated by reference into this written description.

Accordingly, the specific embodiments of the present invention as set forth are not intended as being exhaustive or limiting of the teachings. The scope of the teachings should, therefore, be determined not with reference to this description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventors did not consider such subject matter to be part of the disclosed inventive subject matter.

The disclosures of all articles and references, including patent applications and publications, are incorporated by reference for all purposes.

Plural elements or steps can be provided by a single integrated element or step. Alternatively, a single element or step might be divided into separate plural elements or steps.

The disclosure of “a” or “one” to describe an element or step is not intended to foreclose additional elements or steps.

While the terms first, second, third, etc., may be used herein to describe various elements, components, regions, layers, and/or sections, these elements, components, regions, layers, and/or sections should not be limited by these terms. These terms may be used to distinguish one element, component, region, layer, and/or section from another element, component, region, layer, and/or section. Terms such as “first,” “second,” and other numerical terms when used herein do not imply a sequence or order unless clearly indicated by the context. Thus, a first element, component, region, layer, and/or section discussed below could be termed a second element, component, region, layer, and/or section without departing from the teachings.

The use of “about” or “approximately” in connection with a range applies to both ends of the range. Thus, “about 20 to 30” is intended to cover “about 20 to about 30”, inclusive of at least the specified endpoints.

Unless otherwise stated, any numerical values recited herein include both endpoints and all values from the lower value to the upper value in increments of one unit provided that there is a separation of at least 2 units between any lower value and any higher value. As an example, if it is stated that the amount of a component, a property, or a value of a process variable such as, for example, temperature, time, and the like is, for example, from 1 to 90, from 20 to 80, or from 30 to 70, it is intended that intermediate range values such as (e.g., 15 to 85, 22 to 68, 43 to 51, 30 to 32, etc.) are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner.

The terms “generally” or “about” to describe numbers or numerical ranges may mean±0.2 for numbers from 0.1 to 1, ±2 for numbers from 2 to 100, and ±20 for numbers greater than 100. The foregoing is applicable to all percentages, temperatures, times, surface power densities, or otherwise, unless otherwise stated herein.

The term “consisting essentially of” to describe a combination shall include the elements, components, or steps identified, and such other elements, components, or steps that do not materially affect the basic and novel characteristics of the combination. The use of the terms “comprising” or “including” to describe combinations of elements, components, or steps herein also contemplates embodiments that consist essentially of the elements, components, or steps.

What is claimed is:

1. A method for providing thermal contrast therapy to a vehicle occupant, the method comprising:
first operating a heating device to apply a first temperature for a first period of time to bring a temperature of at least one section of a seat surface to about 43° C. to 48° C. and/or a skin temperature of the vehicle occupant in a region of the at least one section to about 36° C. or more; and
second operating the heating device and a cooling device in an alternating manner for a plurality of time intervals, during the plurality of time intervals the heating device is operative for a second period of time, and the cooling device is operative for a third period of time;
wherein the thermal contrast therapy achieves about a 10° C. to 20° C. change in the temperature of the at least one section of the seat surface and/or about a 3° C. to 6° C. change in the skin temperature of the vehicle occupant in the region of the at least one section of the seat surface.

2. The method according to claim 1, wherein during the plurality of time intervals the heating device and the cooling device effectuate temperature changes in the at least one section of the seat surface; wherein the at least one section includes a lower section, a middle section, an upper section, or any combination thereof.

3. The method according to claim 2, wherein the heating device is located in the lower and middle sections and during the plurality of time intervals the heating device in the lower section is brought to a target temperature of about 63° C. to 67° C. and the heating device in the middle section is brought to a target temperature of about 77° C. to 83° C.

4. The method according to claim 3, wherein the heating device is located in the upper section and during the plurality of time intervals the heating device in the upper section is brought to a target temperature of about 70° C.

5. The method according to claim 3, wherein during the plurality of time intervals, the cooling device is brought to a target temperature of about 18° C. to 22° C.

6. The method according to claim 5, wherein during the plurality of time intervals, the heating device is operated by pulse width modulation with a duty cycle of about 55% to 65% and the cooling device is operated by pulse width modulation with a duty cycle of about 85% to 95%.

7. The method according to claim 6, wherein the operating time of the heating device and the cooling device, during the plurality of time intervals, are equal.

8. The method according to claim 7, wherein the rate of temperature change during the plurality of time intervals is ±0.4° C./minute to ±0.9° C./minute for the skin temperature and/or ±1° C./minute to ±2.5° C./minute for the seat temperature.

9. The method according to claim 8, wherein the plurality of time intervals persist for a duration of about 20 to 35 minutes.

10. The method according to claim 9, wherein the alternating heating and cooling are respectively characterized by a ramp-and-step profile, the ramp-and-step profile of heating being inverse to the ramp-and-step profile of cooling.

11. The method according to claim 10, wherein there is no idle period between the alternating operation of the heating device and the cooling device; wherein the idle period is characterized by a period of time in which both the heating device and the cooling device are not operational.

12. The method according to claim 11, wherein the time intervals include 6 to 16 time intervals.

13. The method according to claim 12, wherein the second period of time is 3 to 5 minutes;
and wherein the third period of time is 3 to 6 minutes.

14. The method according to claim 1, wherein the heating device is operated by pulse width modulation with a duty cycle of about 55% to 65% to realize the first temperature.

15. The method according to claim 14, wherein the first temperature is about 55° C. to 90° C.

16. The method according to claim 15, wherein the first period of time is about 5 to 10 minutes.

17. The method according to claim 1, wherein the thermal contrast therapy is adapted to relieve pain of the vehicle occupant.

18. A device for performing the method according to claim 1, the device comprising: the heating device located in an upper section of the seat, a middle section of the seat, and a lower section of the seat; and a cooling device acting upon the middle section of the seat.

19. The device according to claim 18, wherein the heating device comprises a resistance element; and the cooling device comprises a blower, a thermoelectric device, a fluid distribution device, or any combination thereof; wherein the heating device in the lower section has a surface power density of about 2,100 W/m² to 2,500 W/m², the heating device in the middle section has a surface power density of about 1,900 W/m² to 2,300 W/m², and the heating device in the upper section has a surface power density of about 900 W/m² to 1,500 W/m².

20. A vehicle seat comprising the device according to claim 18.
